(19) United States
(12) Patent Application Publication
(10) Pub. No.: US 2023/0172785 A1
(43) Pub. Date: Jun. 8, 2023

(54) PENIS STRETCHER AND METHOD FOR TRAINING PENIS

(71) Applicant: Hui LI, Jiangsu (CN)

(72) Inventor: Hui LI, Jiangsu (CN)

(21) Appl. No.: 17/919,417

(22) PCT Filed: Jun. 11, 2021
(86) PCT No.: PCT/CN2021/099607

§ 371 Date: Oct. 17, 2022

(30) Foreign Application Priority Data
Apr. 17, 2020 (CN) 202010303761.X

(51) Int. Cl. A61H 1/02; A61H 19/00; A61H 9/00

(52) U.S. Cl. CPC A61H 1/0218; A61H 19/30; A61H 9/0057; A61H 2205/087; A61H 2201/5005; A61H 2201/1207; A61H 2201/1246

(57) ABSTRACT

A device and a training method used for stretching the male penis, includes a penis tip component, an elastic component, and a varying force adding component, and able to exert a cyclically varying pulling force on the penis to perform exercising. Male persons can achieve the goal of increasing the size of the penis by using the present device for training. Device and method can be used to solve the problem of the male reproductive organ being short and small.

### TECHNICAL FIELD

The present invention relates to an exercise device used to exercise and enlarge penis and an exercise method.

### BACKGROUND ART

Penis is an important organ of the male urinary and reproductive system. A healthy penis can not only perform normal physiological functions, but also play an important role in enhancing male self-confidence. Studies have shown that the average length of Chinese male penis is 8 cm in a weak state, and 11-16 cm after erection. If it is less than 5 cm in a weak slate and less than 8 cm when erected, it is considered to be short penis. A short penis will cause disharmony in the family relationship between husband and wife and cause great pressure on men's psychology.

At present, methods for solving short penis include technologies such as penis lengthening surgery and penis exercise equipment etc. The effect of penis lengthening surgery is not ideal, and it is easy to damage the tissue structure of the penis, and cause loss of sexual function in severe cases.

Penis stretching device is a type of penis exercise equipment, and its principle is basically stretching the penis and exercising the penis by using a constant pulling force, such as the traditional plan of stretching the penis by hanging a weight. This type of exercise is not ideal, the blood circulation alter the penis is stretched is a big problem. If the time is too long, it is easy to cause ED due to poor blood circulation, and the effect of penis enlargement is very small.

### SUMMARY OF THE INVENTION

The purpose of the present invention is providing a penis exercise device and an exercise method which are better than the existing penis stretching exercise equipment and methods, so that the effect of penis enlargement is better, and the problem of short penis in patients with short penis can also be solved.

The purpose of the present invention is achieved as follows: a penis stretcher is provided for stretching and exercising penis to enlarge penis, comprising a penis-end part which can be placed on the penis, characterized in that: the penis stretcher also has an elastic part, one end of the elastic part is connected to the penis-end part, and the other end of the elastic part (in use) may be connected to a force applying part. The term “connected” herein includes direct connection and indirect connection; force applying part is an optional item, which may or may not be comprised. In use, the force applying part is needed.

The terms “connection” and “connected” herein include both direct and indirect connections.

The above-mentioned expression “may be connected to a force applying part” that when manufacturing and selling products based on the present invention, a force applying part may be provided; or the force applying part may not be provided and may be replaced by the user with water-containing liquid containers, metal heavy objects, other heavy objects or other force applying parts at home. That is to say, when the product is manufactured and sold, whether it comprises force applying part or not, as long as it can be connected with a force applying part, it belongs to the protection scope of the present invention.

The elastic part may be items made of common materials with better elasticity such as springs, rubber bands etc.

The penis-end part may enclose the surface of the penis, and the contact surface surrounding the penis can shrink.

The penis-end part may be a vacuum suction adsorption device like a vacuum cupping device, or a device that can shrink by pulling after forming a circle like a trouser belt, and it may also be a device that can shrink to the center axis of the penis and is formed by a plurality of movable blocks working together around the penis, and it may also be a device that can clamp the penis by inflating a hollow annular container to expand it inwardly, or other devices that can shrink toward the center.

The penis-end part is any part or device that can enclose a hole, wherein the penis can pass through the hole, and the cross-sectional area of the hole can be reduced.

The penis-end part can be fixed to the penis.

When in use, the penis-end part is fixed near coronary sulcus of the penis.

When in use, the other end of the elastic part can be used to connect with the force applying part.

The force applying part can be a heavy object applying force by gravity, or a pan or device that can move relative to the human body so as to apply force to the penis.

A penis stretcher is provided for stretching and exercising the penis to enlarge the penis, comprising a penis-end pan which can be placed on the penis, wherein: the penis stretcher also has a force applying part (varying force applying part) which is connected (directly or indirectly) to the penis-end part, and when in use, the force applying part can apply a varying pulling force to the penis, so that the inside of the penis is subjected to alternating tensile stress. The “varying force applying part” refers to parts that can apply varying forces on other objects.

Preferably, the above-mentioned varying pulling force varies cyclically.

Preferably, the mass of the force applying part is at least 200 grams.

Preferably, the mass of the force applying part is at least 500 grams.

Preferably, the mass of the force applying part is at least 1,000 grams.

As an improvement, the penis stretcher also comprises a force applying part which is connected to the other end of the elastic part.

The force applying pan can apply a varying pulling force to the penis through the elastic part, so that the inside of the penis is subjected to alternating tensile stress.

Preferably, the force applying part comprises an eccentric motor.

Preferably, the force applying part comprises an electromagnet.

Preferably, the force applying part comprises an electromagnetic motor that can realize linear motion.

Preferably, the force applying part comprises any mechanical motion mechanism that can be driven by electromotor, such as a four-bar linkage mechanism, a crank linkage mechanism, and a crank slider mechanism, etc., and for details, please refer to textbooks on mechanical principles or existing technical materials.

Preferably, the force applying part comprises a vacuum-pumping mechanism.

Preferably, the force applying part comprises a pneumatic or hydraulic mechanism.

As an improvement, the penis stretcher also has a control part to control the change amplitude, frequency, on-off, etc. of the output force of the force applying part.

Any of the above-mentioned penis stretchers comprises constant force applying part.

In any of the above-mentioned penis stretchers, the force applying part is composed of constant force applying pan and varying force applying part. Constant force applying means that the applied force is constant during one use. With the strengthening of exercise, a constant but greater force can also be used. The varying force applying part means that the force applied to the penis by the force applying part varies during one use.

Preferably, the force applied by the above-mentioned force applying part and varying force applying part varies cyclically.

The above-mentioned fixing position may be any position on the penis, such as the root, the middle or the coronary sulcus of the glans.

The above-mentioned penis-end part and elastic part may be (undertaken by) the same part.

Any one of the above-mentioned penis exercisers causes the inside of the penis to be subjected to alternating tensile stress.

A method for exercising penis is provided, wherein: the penis is exercised by applying a varying force to the penis.

Preferably, the force varies cyclically.

Preferably, the force is pulling force. This pulling force can subject the inside of penis to varying tensile stress.

Preferably, the force is pressure, bending force, or torsion.

Preferably, the pulling force, pressure, bending force, and torsion may all vary cyclically. The inside of the penis is subjected to alternating stress.

A penis exercise device is provided for enlarging the penis, wherein: it has a varying force applying part that can exert a varying (especially continuously varying) force on the penis.

Preferably, the varying force applying part can exert cyclically varying force on the penis.

Preferably, the force is continuously varying.

Preferably, the force may be pulling force, pressure, bending force or torsion.

Preferably, a constant force applying part is also provided.

Preferably, the mass of the constant force applying part is at least 200 grams.

Preferably, the mass of the constant force applying part is at least 500 grams.

Preferably, the mass of the constant force applying part is at least 1,000 grams.

For device composed of penis-end parts, elastic parts, and hanging gravity parts, the user can move the gravity parts by their own ups and downs or manually, causing the gravity parts to reciprocate (vibrate), thereby causing the inside of the penis to be subjected to alternating tensile stress.

In order to better count and master the exercise situation, the penis stretcher can be equipped with a pulling force sensor or a pressure sensor for measuring the force and intensity of pressure on the penis during the exercise. These values can be displayed on the LCD screen after calculated by the control components.

Preferably, it has a wireless communication module for wireless connection and communication with the mobile phone to display relevant data on the mobile phone.

The wireless communication module may be an infrared module, Bluetooth module, WIFI or other wireless transceiver module, which can send data during exercise to the user's smart phone, so that the related data (such as pulling force, pressure, intensity of pressure, variation of pulling force, exercise time, amplitude of force variation, frequency of force variation, etc.) are displayed in the supporting mobile phone APP.

The force applying part may be driven by storage batteries such as lithium batteries, or may be directly plugged in for use.

When the required alternating pulling force is generated by rotating the eccentric motor, the amplitude and frequency of the pulling force variation can be adjusted by adjusting the electromotor speed. The amplitude of the pulling force variation can also be adjusted by adjusting the distance between the eccentric block on the eccentric motor and the rotating shaft.

When a linear electromagnet is used, the amplitude and the frequency of the pulling force variation can be changed by adjusting the voltage. The electromagnet drives the counterweight block for reciprocating motion to produce alternating tensile stress.

Vacuum traction can be used in a force applying device to provide pulling force, and cyclic alternating stress can be realized by changing the vacuum degree.

The force applying part can also be a common vibration mechanism.

Advantages: the exercise device and method of the present invention exercise the penis with varying force, so that the inside of the penis is subjected to alternating tensile stress. During the stretching exercise of the penis, the blood circulation of the penis can be promoted and more nutrition can be brought to the penis, not only the effect of penis enlargement is better, but also the problem of ED caused by weight-hanging exercise can be avoided, and it is also safer and more reliable than penis enlargement surgery.

### BRIEF DESCRIPTION OF DRAWINGS

The present invention will be further described below in conjunction with the drawings and examples.

FIG. 1 is a three-dimensional view of example 1 of the present invention.

FIG. 2 is a three-dimensional perspective view of alternating power generator 4 in FIG. 1.

FIG. 3 is a three-dimensional view of example 2 of the present invention.

FIG. 4 is a three-dimensional view of example 3 of the present invention.

FIG. 5 is a three-dimensional view of example 4 of the present invention.

FIG. 6 is a three-dimensional perspective view of penile-end part in examples 1, 2, 3, and 4 of the present invention.

FIG. 7 is a three-dimensional view of example 5 of the present invention.

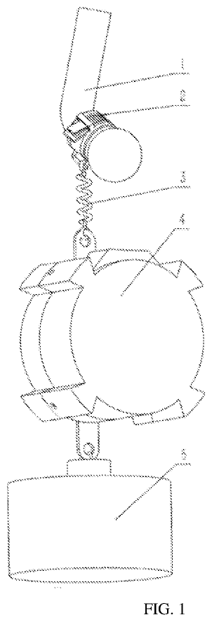

In FIG. 1: 1, penis; 2, penis-end part; 3, spring; 4, alternating power generator; 5, counterweight block;

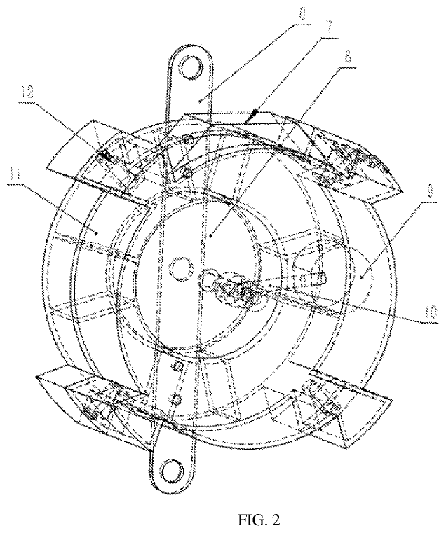

In FIG. 2: 6, connecting plate; 7, LCD screen; 8, electromotor; 9, eccentric block; 10, connecting rod; 11, shell; 12, shell;

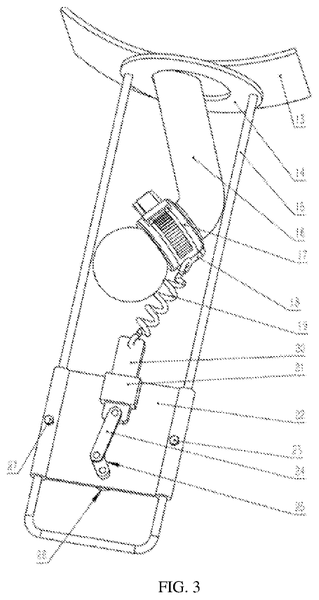

In FIG. 3: 13, human abdomen; 14, baffle plate; 15, sliding rail frame; 16, penis; 17, penis-end part; 18, hanging ring; 19, spring; 20, guide rod; 21, guide rod support ; 22, carriage; 23, positioning bolt; 24, connecting rod; 25, crank; 26, electromotor; 27, positioning bolt;

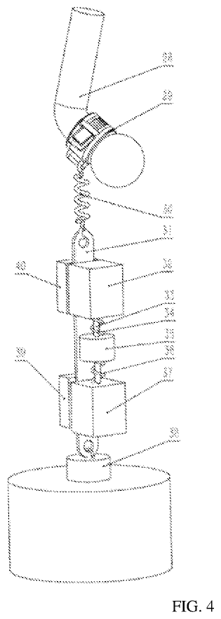

In FIG. 4: 28, penis; 29, penis-end part; 30, spring; 31, connecting plate; 32, electromagnet; 33, guide rod; 34, spring; 35, counterweight block; 36, spring; 37, electromagnet; 38, counterweight block; 39, lithium battery; 40, control module;

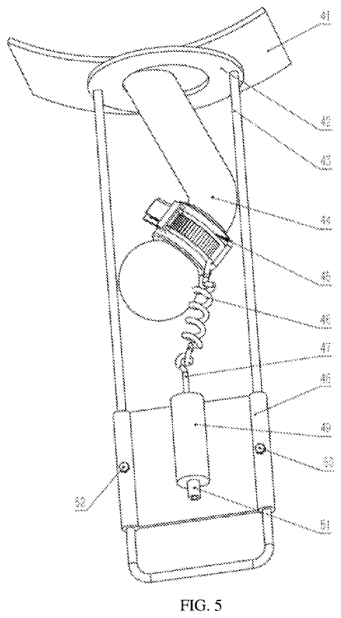

In FIG. 5: 41, human abdomen; 42, baffle plate; 43, sliding rail frame; 44, penis; 45, penis-end part; 46, spring; 47, piston pull rod; 48, carriage; 49, piston assembly; 50, positioning bolt; 51, conduit; 52, positioning bolt.

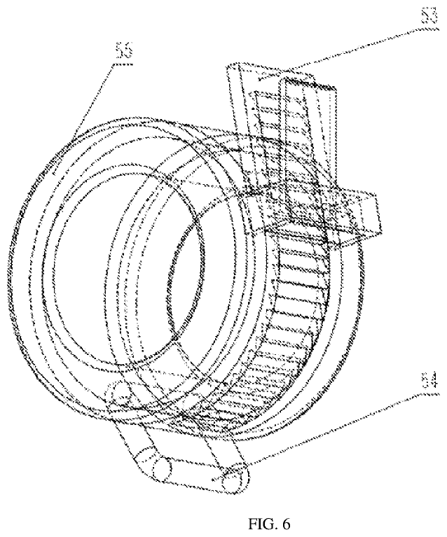

In FIG. 6: 53, belt; 54, hanging ring; 55, soft rubber ring.

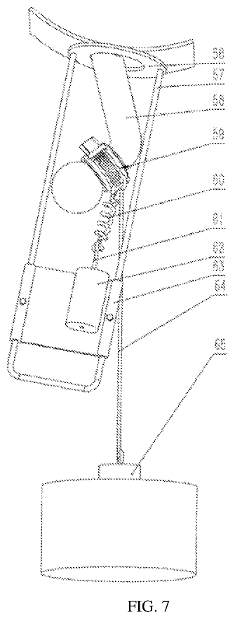

In FIG. 7: 56, baffle plate; 57, sliding rail frame; 58, penis; 59, penis-end part; 60, spring; 61, pull rod; 62, electromagnet assembly; 63, carriage; 64, rope; 65, counterweight block.

### EMBODIMENTS

Example 1 shown in FIG. 1 is an embodiment of the present invention.

In FIG. 1, the penis-end part 2 is fixed on the penis stem near the coronary sulcus of the glans by clamping, the upper end of the spring 3 is connected with the hanging ring (the hanging ring 54 in FIG. 6) on the penis-end part 2, and the lower end of the spring is connected to the hole on the connecting plate (part 6 in FIG. 2) on the alternating power generator 4, and the lower end of the connecting plate 6 of the alternating power generator 4 is connected with the counterweight block 5.

In FIG. 2, the connecting plate 6 is fixedly installed on the back of the shell 12, and the shell 11 and the shell 12 are fixedly installed together. The LCD screen 7 is installed on the shell 11, and a control circuit board is installed below. The electromotor 8 is installed inside the shell 12, and the eccentric block 9 is fixedly connected to the rotating shaft of the electromotor 8 through the connecting rod 10. The rotation of the electromotor 8 can drive the eccentric block 9 to rotate, thereby generating a varying force in the vertical direction.

In FIG. 6, the soft rubber ring 55 is sleeved around the penis, the belt 53 surrounds the groove on the soft rubber ring 55 from the outside, the belt 53 passes through the center of the hanging ring 54, and the upper part of the hanging ring 54 is clamped between the belt 53 and the soft rubber ring 55, the belt 53 can be clamped and fixed to the penis after being tightened. The function of the soft rubber ring 55 is to make the clamped part of the penis more comfortable.

The gravity of the counterweight block 5 is constant, and the alternating power generator 4 generates a cyclically varying pulling force in the vertical direction. The two forces work together to act on the penis, causing the inside of the penis to be subjected to alternating tensile stress.

The amplitude and the variation frequency of the pulling force acting on the penis can be adjusted by adjusting the rotation speed of the electromotor 8. The pulling force can also be adjusted by adjusting the weight of the eccentric block 9 or the distance between the eccentric block and the rotating shaft of the electromotor 8.

The upper and lower positions of the alternating power generator 4 and the counterweight block 5 can also be interchanged, or they can be fixed together.

In this example: the spring 3 is an elastic part, the alternating power generator 4 and the counterweight block 5 constitute a force applying part, and the alternating power generator 4 is a varying force applying part.

Example 2 shown in FIG. 3 is another embodiment of the present invention.

The baffle plate 14 is pressed against the abdomen of the human body, and the penis 16 passes through the central hole of the baffle plate 14. The sliding rail frame 15 and the baffle plate 14 are fixed together. The sliding rail frame 15 passes through the hole on the carriage 22, and the carriage 22 can move on the sliding rail frame 15. The positioning bolts 23 and 27 can fix the position of the carriage 22 on the sliding rail frame 15 to adapt to penises of different lengths. The penis-end part 17 is fixed on the penis stem near the coronary sulcus of the glans, its lower end is connected with the spring 19, and the other end of the spring is connected with the hole on the guide rod 20. The guide rod can reciprocate in the hole of the guide rod support 21. The guide rod support 21 is installed on the carriage 22. The upper end of the connecting rod 24 is hinged with the lower end of the guide rod 20. One end of the crank 25 is hinged with the lower end of the connecting rod 24, and the other end of the crank 25 is fixedly connected with the rotating shaft of the electromotor 26.

When the rotating shaft of the electromotor 26 rotates, it can drive the crank 25 to rotate and drive the connecting rod 24 and the guide rod 20 to move. The up and down movement of the guide rod 20 pulls the spring 19, so that the penis 16 is subjected to a downward cyclically varying pulling force.

The amplitude of the pulling force acting on the penis can be adjusted by adjusting the position of the carriage 22. The variation frequency of the pulling force acting on the penis can be adjusted by adjusting the rotation speed of the electromotor.

In this example: the spring 19 is an elastic part, and the parts 14, 15, 20-27 collectively constitute a varying force applying part.

Example 3 shown in FIG. 4 is another embodiment of the present invention.

The penis-end part 29 is fixed on the penis stem near the coronary sulcus of the glans, its lower end is connected with the spring 30, and the other end of the spring 30 is connected with a hole on the connecting plate 31. The electromagnets 32 and 37 are fixed on the connecting plate 31. The counterweight block 35 is fixed on the guide rod 33, and the guide rod 33 can move up and down as being attracted by electromagnetic force in the electromagnets 32 and 37. The counterweight block 35 and the electromagnets 32, 37 are directly installed with springs 34, 36, respectively, and the springs can buffer the collision between them, transfer force, and avoid noise. The lower end of the connecting plate 31 is connected to the counterweight block 38. The lithium battery 39 and the control module 40 are connected by electric wire, and they are both fixedly installed on the connecting plate 31. The control module 40 and the electromagnets 32 and 37 are connected by electric wire to control the energization and de-energization of the electromagnet. When the electromagnets 32 and 37 are energized and de-energized alternately, the counterweight block 35 can move up and down, so that the pulling force acting on the penis varies.

In this example: the spring 30 is an elastic part; the parts 31-40 collectively constitute a force applying part (varying force applying part).

Example 4 shown in FIG. 5 is another embodiment of the present invention.

Example 4 is different from example 2 shown in FIG. 3 in that: a different force applying device is used here. The lower end of the spring 46 is connected with the piston rod 47, and the piston rod 47 is connected with a piston column inside the piston assembly 49. Therefore, the movement of the piston column can make the piston rod 47 move up and down, so that the penis is subjected to varying pulling force. One end of the conduit 51 on the piston assembly 49 is connected to the piston cavity, and the other end is connected to a vacuum suction device (omitted here for ease of drawing). Changing the vacuum degree generated by the vacuum suction device can cause the reciprocating motion of the piston rod 47.

In this example: the spring 46 is an elastic part; the parts 42, 43, 47-52, and the vacuum suction device collectively constitute a (varying) force applying part. Here, a counterweight block generating a constant pulling force can also be used in combination with the above-mentioned force applying part, and the counterweight block is directly connected with the penis-end part, so that the power consumption of the vacuum suction device can be reduced.

In this example, the piston rod may also be directly connected to the penis-end part (without the spring).

Example 5 shown in FIG. 7 is another embodiment of the present invention.

Example 5 is different from example 4 in that: the piston assembly 49 of example 4 is replaced with an electromagnet assembly 62, the pulling force of the pull rod 61 is directly controlled by the electromagnet, and the magnitude of the pulling force is controlled by the current and voltage on the electromagnet, a control part can control the voltage and current loaded on the electromagnet as well as their changes. These changes will eventually lead to changes in the pulling force acting on the penis. The counterweight block 65 provides a constant pulling force cooperating with the electromagnetic pulling force; the counterweight block 65 is connected to the penis-end part 59 through a rope 64, so as to reduce the power consumption of the electromagnetic force applying device.

In this example, the pull rod 61 can also be directly connected to the penis-end part 59 (without the spring).

In this example: the spring 60 is an elastic part; the parts 56, 57, and 61-65 collectively constitute (varying) force applying part.

In order to prevent the electromagnet from affecting the human body, an electromagnetic shielding device can be installed between the electromagnet and the human body.

For ease of drawing, common matched parts that can be completed by those skilled in the art, such as air valves, vacuum pumping devices, control parts, control circuits, electric wires, power sources (common power sources such as storage batteries, dry batteries, direct plug-in power sources, etc. can be used), and control devices, are omitted here, but it does not mean that there are no such devices.

The terms such as part, component, parts and components mentioned here have no special restrictions or special definitions or meanings on the number and relationship of the contained parts, and are not limited to the concept of mechanical textbooks. The concepts such as equipment, instrument, and device, etc. described herein have no special requirements on the number or complexity of the contained parts, and are not limited by the concept of textbooks.

1. A penis stretcher, used for stretching and exercising penis to enlarge penis, comprising a penis-end part which can be placed on the penis, wherein, the penis stretcher also has an elastic part, one end of the elastic part is connected to the penis-end part, and the other end of the elastic part may be connected to a force applying part.

2. A penis stretcher, used for stretching and exercising penis to enlarge penis, comprising a penis-end part which can be placed on the penis, wherein, the penis stretcher also has a varying force applying part which is connected (directly or indirectly) to the penis-end part, and when in use, the varying force applying part can apply a varying pulling force to the penis.

3. The penis stretcher according to claim 2, wherein the force applied by the varying force applying part varies cyclically.

4. The penis stretcher according to claim 1, wherein the force applying part and the varying force applying part comprise an electromagnet.

5. A method for exercising penis, wherein the penis is exercised by applying a varying force to the penis.

6. The method for exercising penis according to claim 5, wherein the force varies cyclically.

7. The penis stretcher according to claim 2, wherein a constant force applying part is also provided.

8. The penis stretcher according to claim 7, wherein a constant force

9. The penis stretcher according to claim 1, wherein the force applying part comprises a varying force applying part.

10. The penis stretcher according to claim 1, wherein the force applying part comprises a varying force applying part and a constant force applying part.

11. The penis stretcher according to claim 2, wherein the force applying part and the varying force applying part comprise an electromagnet.
